(19) United States
(12) Patent Application Publication
(10) Pub. No.: US 2007/0249563 A1
(43) Pub. Date: Oct. 25, 2007

(54) PROCESS FOR PREPARING FOSPHENYTOIN

(76) Inventors: Apurba Bhattacharya, Hyderabad (IN); Vijayabhaskar Bolugoddu, Hyderabad (IN); Pravinchandra Jayantilal Vankawala, Hyderabad (IN); Chandrasekhar Ravi Ram Elati, Hyderabad (IN); Srinivas Gangula, Hyderabad (IN); Amarnath Reddy Lekkala, Hyderabad (IN); Ramakrishna Venkata Mallemula, Kadapa (IN); Anitha Naredla, Karimnagar (IN); Ashok Sigala, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/737,783

(22) Filed: Apr. 20, 2007

Related U.S. Application Data
(60) Provisional application No. 60/820,838, filed on Jul. 31, 2006.
(60) Provisional application No. 60/821,444, filed on Aug. 4, 2006.

(30) Foreign Application Priority Data
Apr. 21, 2006 (IN) 734/CHE/2006
Jun. 14, 2006 (IN) 1031/CHE/2006

(51) Int. Cl. A61K 31/675; C07F 9/6506

(52) U.S. Cl. 514/94; 548/112

(57) ABSTRACT

Processes for preparing fosphenytoin.

### INTRODUCTION TO THE INVENTION

The present invention relates to processes for the preparation of fosphenytoin and its pharmaceutically acceptable salts. In addition, the present invention relates to intermediate compounds that can be used in the preparation of fosphenytoin and its pharmaceutically acceptable salts.

Fosphenytoin sodium has a chemical name 5,5-diphenyl-3-[(phosphono-oxy)methyl]imidazolidine-2,4-dione disodium salt, and is structurally represented by Formula I.

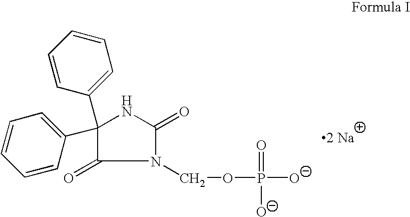

Fosphenytoin sodium is useful as an anticonvulsive, antiepileptic and antiarrhythmic agent. It is available in the market under the brand name CEREBYX® in vials for intravenous administration containing 75 mg/ml of fosphenytoin sodium, equivalent to 50 mg/ml phenytoin sodium after administration. Fosphenytoin is a prodrug that is metabolized to the active agent phenytoin.

U.S. Pat. No. 4,260,769 discloses fosphenytoin and its related compounds along with their pharmaceutically acceptable salts. The patent also gives a process for the preparation of derivatives of fosphenytoin and their salts.

U.S. Pat. Nos. 6,022,975 and 6,255,492 disclose processes for preparation of fosphenytoin sodium and its homologues.

Processes for the preparation of fosphenytoin and its salts have also been described in U.S. Patent Application Publication No. 2005/0272706 A1, and Drugs of the Future, Vol.14, No. 7, pages 611 to 613, 1989.

There is a need to provide a simple, eco-friendly, industrially feasible, cost effective, and robust process for the synthesis of fosphenytoin and its salts in high yield and purity.

The present invention provides processes for the preparation of fosphenytoin which involve a reduced number of stages, and can be practiced on an industrial scale. An advantage of the processes of the present invention is that all synthetic steps are performed under mild conditions providing a low content of by-products and thereby a high yield and high purity of the final product.

### SUMMARY OF THE INVENTION

The present invention relates to processes for preparation of fosphenytoin and its pharmaceutically acceptable salts. It also relates to new intermediates which can be used in the preparation of fosphenytoin and its pharmaceutically acceptable salts.

One aspect of the present invention provides a process for the preparation of fosphenytoin and its pharmaceutically acceptable salts. In an embodiment, the process comprises:

a) reacting 3-hydroxymethyl-5,5-diphenyl-2,4-imidazolidinedione of Formula II with trichloroacetonitrile in the presence of a suitable base to give 2,2,2-trichloro-acetimidic acid 2,5-dioxo-4,4-diphenyl-imidazolidin-1-ylmethyl ester of Formula III;

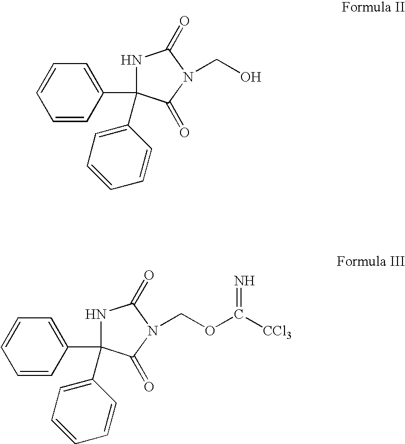

b) reacting 2,2,2-trichloro-acetimidic acid 2,5-dioxo-4,4-diphenyl-imidazolidin-1-ylmethyl ester of Formula III with dibenzyl phosphate to afford phosphoric acid dibenzyl ester 2,5-dioxo-4,4-diphenyl-imidazolidin-1-yl methyl ester of Formula IV; and

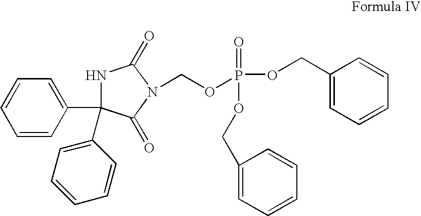

c) reacting dibenzyl ester 2,5-dioxo-4,4-diphenyl-imidazolidin-1-ylmethyl ester of Formula IV with a suitable reagent to afford fosphenytoin of Formula V.

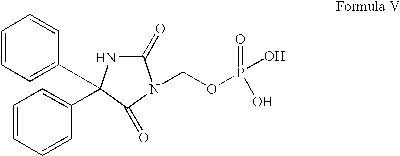

A further aspect of the present invention provides another process for the preparation of fosphenytoin and its pharmaceutically acceptable salts. In an embodiment, the process comprises:

a) reacting 5,5-diphenyl-imidazolidine-2,4-dione of Formula VI with formaldehyde in the presence of a suitable base to afford 3-hydroxymethyl-5,5-diphenyl-imidazolidine-2,4-dione of Formula II;

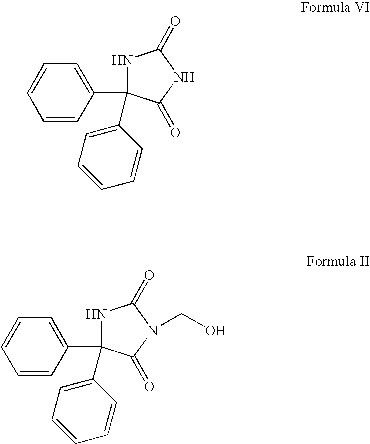

b) reacting 3-hydroxymethyl-5,5-diphenyl-imidazolidine-2,4-dione Formula II with 3,4-dihydro-2H-pyran of Formula VII in the presence of a suitable base to give 5,5-diphenyl-3-(tetrahydro-pyran-2-yloxymethyl)-imidazolidine-2,4-dione of Formula of VIII;

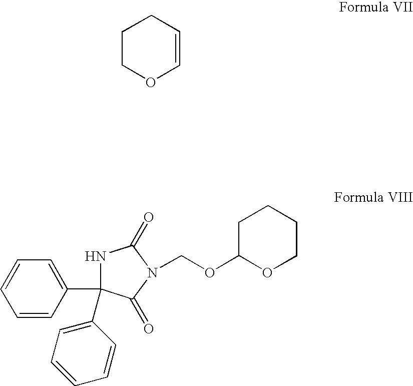

c) reacting 5,5-diphenyl-3-(tetrahydro-pyran-2-yloxymethyl)-imidazolidine-2,4-dione of Formula VIII with benzyl bromide in the presence of a suitable base to give 1-benzyl-3-(3,4-d ihydro-2H-pyran-2-yloxymethyl)-5,5-diphenyl-imidazolidine-2,4-dione of Formula IX;

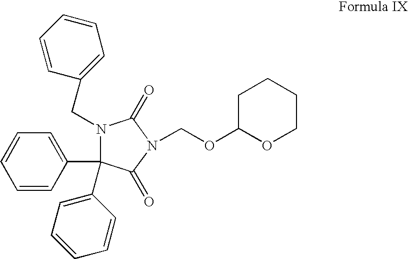

d) reacting 1-benzyl-3-(3,4-dihydro-2H-pyran-2-yloxymethyl)-5,5-diphenyl-imidazolidine-2,4-dione of Formula IX with a suitable acid to give 1-benzyl-3-hydroxymethyl-5,5-diphenyl-imidazolidine-2,4-dione of Formula X;

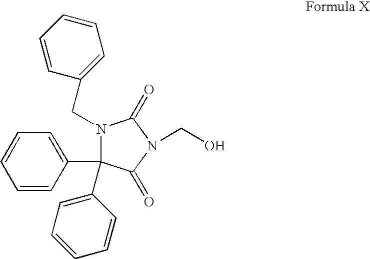

e) reacting 1-benzyl-3-hydroxymethyl-5,5-diphenyl-imidazolidine-2,4-dione of Formula X with a suitable halogenating agent to give 1-benzyl-3-halomethyl-5,5-diphenyl-imidazolidin-3-2,4-dione of Formula XI 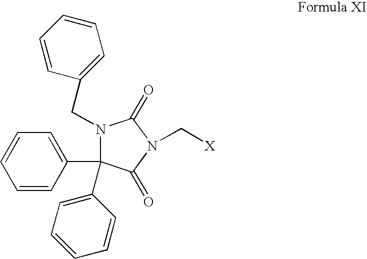
where X is Cl, Br, or I;

f) reacting 1-benzyl-3-halomethyl-5,5-diphenyl-imidazolidin-3-2,4-dione of Formula XI with a dibenzyl phosphate in the presence of a suitable base to give phosphoric acid dibenzyl ester 3-benzyl-2,5-dioxo-4,4-diphenyl-imidazolidin-1-yl methyl ester of Formula XII; and

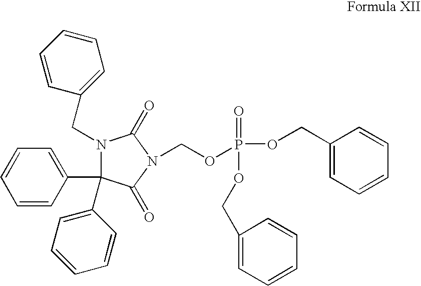

g) reacting phosphoric acid dibenzyl ester 3-benzyl-2,5-dioxo-4,4-diphenyl-imidazolidin-1-ylmethyl ester of Formula XII with a suitable reagent to give fosphenytoin of Formula V.

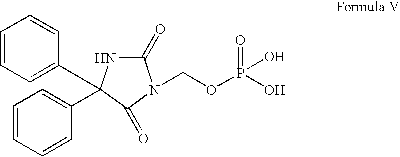

Yet another aspect of the present invention provides a process for the conversion of fosphenytoin to its sodium salt using less than 2.5 moles of sodium hydroxide, per mole of fosphenytoin. In an embodiment, the amount of sodium hydroxide used is about 2 to about 2.5 moles, per mole of fosphenytoin.

Yet other aspects of the present invention provide the following intermediate compounds that are useful for the preparation of fosphenytoin and its pharmaceutically acceptable salts:

a) 2,2,2-trichloro-acetimidic acid 2,5-dioxo-4,4-diphenyl-imidazolidin-1-ylmethyl ester of Formula II;

b) 5,5-diphenyl-3-(tetrahydro-pyran-2-yloxymethyl)-imidazolidine-2,4-dione of Formula VIII;

c) 1-benzyl-5,5-diphenyl-3-(tetrahydro-pyran-2-yloxymethyl)-imidazolidine-2,4-dione of Formula IX;

d) 1-benzyl-3-hydroxymethyl-5,5-diphenyl-imidazolidine-2,4-dione of Formula X;

e) 1-benzyl-3-halomethyl-5,5-diphenyl-imidazolidine-2,4-dione of Formula XI; and

f) phosphoric acid dibenzyl ester 3-benzyl-2,5-dioxo-4,4-diphenyl-imidazolidin-1-yl methyl ester of Formula XII.

Still another aspect of the present invention provides substantially pure fosphenytoin and its pharmaceutically acceptable salts. In an embodiment, the invention provides fosphenytoin or a salt thereof, containing less than about 0.5 weight percent of individual impurities comprising one or more of:

a) a compound having a formula:

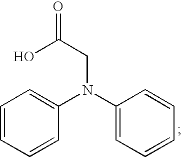

b) a compound having a formula:

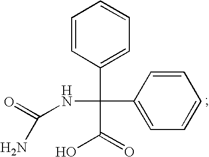

c) a compound having a formula:

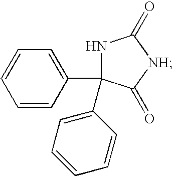

d) a compound having a formula:

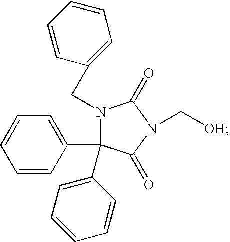

e) a compound having a formula:

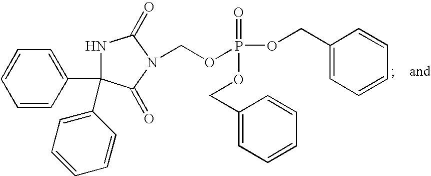

f) a compound having a formula:

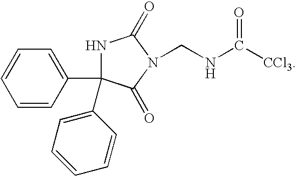

A further aspect of the present invention provides amorphous fosphenytoin sodium and a process for its preparation.

A still further aspect of the present invention provides a pharmaceutical composition comprising fosphenytoin or its pharmaceutically acceptable salts prepared according to the process of the present invention along with one or more pharmaceutically acceptable carriers, excipients or diluents.

In an embodiment, the invention provides a process for preparing fosphenytoin or a salt thereof, comprising reacting a compound having a formula: 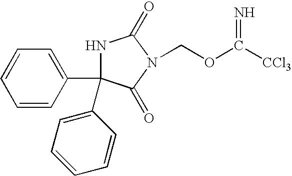
with dibenzyl phosphate, to form a compound having a formula:

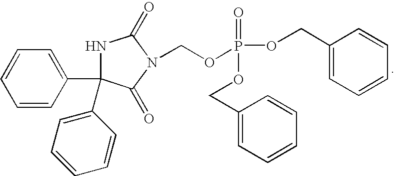

In another embodiment, the invention provides a compound having a formula:

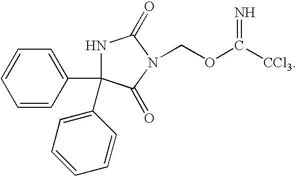

In an additional embodiment, the invention provides a process for preparing fosphenytoin or a salt thereof, comprising reacting a compound having a formula: 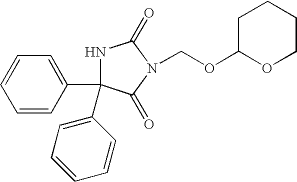
with a benzyl halide, to form a compound having a formula:

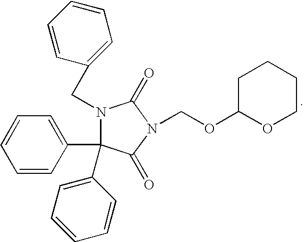

In a further embodiment, the invention provides a process for preparing fosphenytoin or a salt thereof, comprising reacting a compound having a formula: 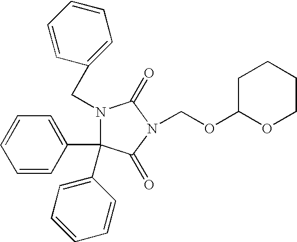
with an acid to form a compound having a formula:

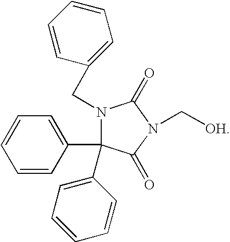

In another embodiment, the invention provides a process for preparing fosphenytoin or a salt thereof, comprising reacting a compound having a formula: 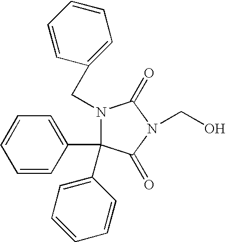
with a halogenating agent to form a compound having a formula: 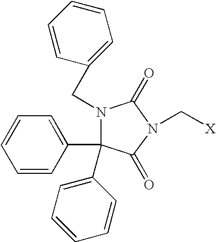
where X is Cl, Br, or I.

An embodiment of the invention provides a process for preparing fosphenytoin or a salt thereof, comprising reacting a compound having a formula: 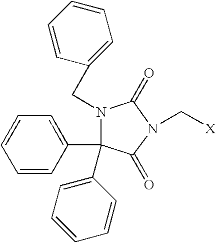
with a dibenzyl phosphate to form a compound having a formula:

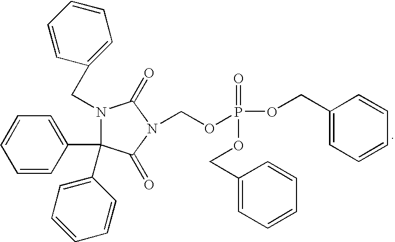

An embodiment of the invention provides a process for preparing fosphenytoin or a salt thereof, comprising reacting a compound having a formula:

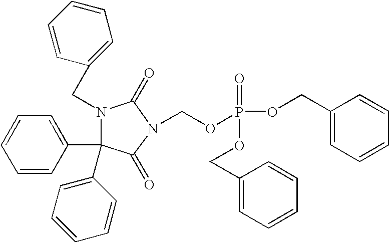

with a reducing agent to form fosphenytoin.

An embodiment of the invention provides a compound having a formula:

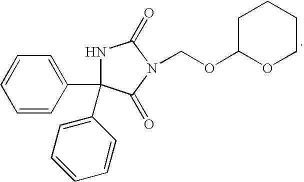

An embodiment of the invention provides a compound having a formula:

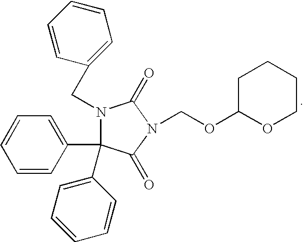

An embodiment of the invention provides a compound having a formula:

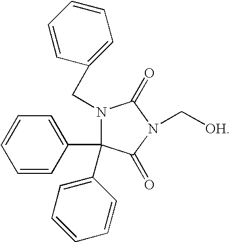

An embodiment of the invention provides a compound having a formula: 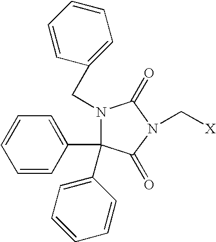
where X is Cl, Br, I.

An embodiment of the invention provides a compound having a formula:

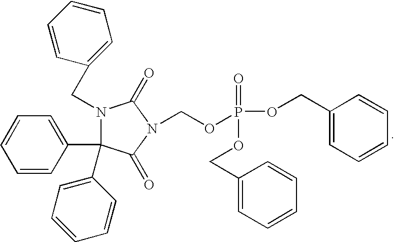

Another embodiment of the invention provides fosphenytoin or a salt thereof, containing less than less than about 2 weight percent of a phosphate salt.

### BRIEF DESCRIPTION OF THE DRAWINGS

FIG. 1 is a schematic representation of a process for the preparation of fosphenytoin starting from the intermediate of Formula II.

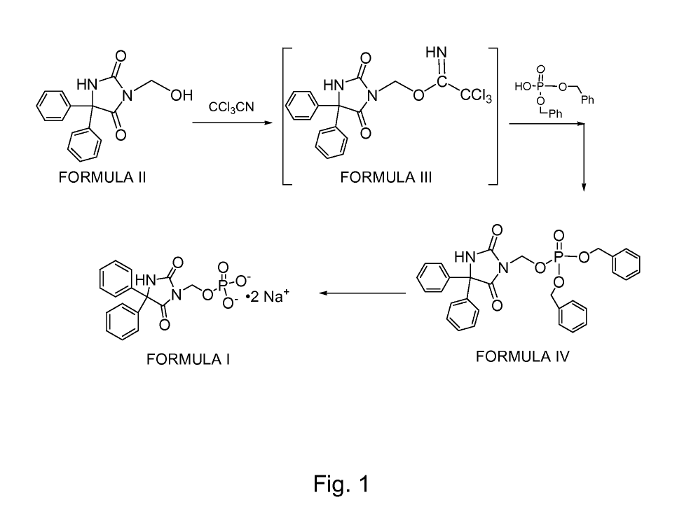

FIG. 2 is a schematic representation of a process for the preparation of fosphenytoin starting from the intermediate of Formula VI.

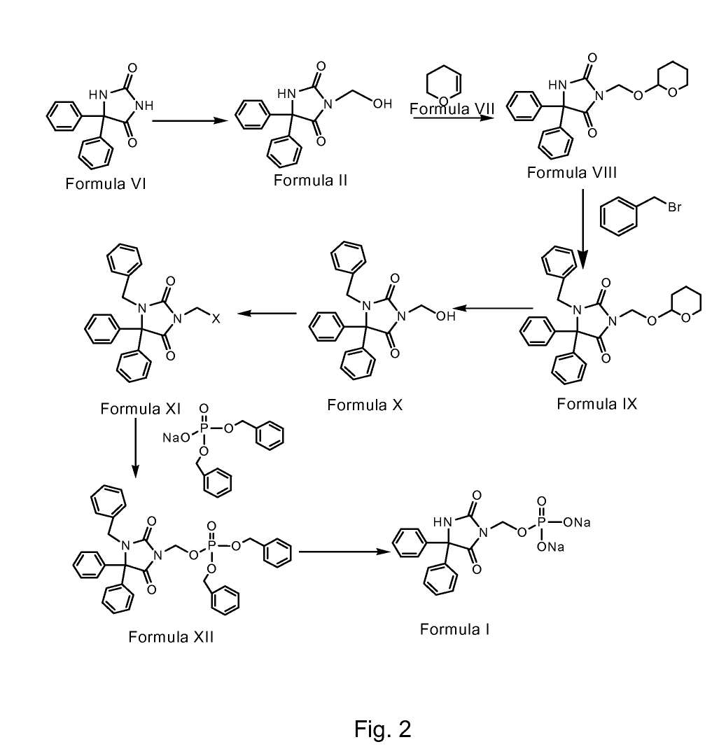

FIG. 3 is an X-ray powder diffraction pattern of crystalline fosphenytoin sodium prepared in Example 7.

FIG. 4 is an X-ray powder diffraction pattern of amorphous fosphenytoin sodium prepared in Example 14.

### DETAILED DESCRIPTION OF THE INVENTION

The present invention relates to processes for preparation of fosphenytoin and its pharmaceutically acceptable salts. It also relates to intermediate compounds that can be used in the preparation of fosphenytoin and its pharmaceutically acceptable salts.

One aspect of the present invention provides a process for the preparation of fosphenytoin and its pharmaceutically acceptable salts. In an embodiment, the process comprises:

a) reacting 3-hydroxymethyl-5,5-diphenyl-2,4-imidazolidinedione of Formula II with trichloroacetonitrile in the presence of a suitable base to give 2,2,2-trichloro-acetimidic acid 2,5-dioxo-4,4-diphenyl-imidazolidin-1-ylmethyl ester of Formula III.

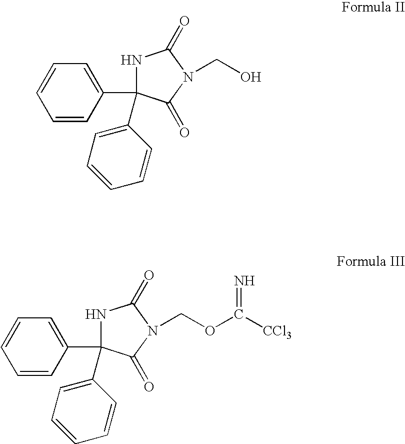

b) reacting 2,2,2-trichloro-acetimidic acid 2,5-dioxo-4,4-diphenyl-imidazolidin-1-ylmethyl ester of Formula III with dibenzyl phosphate to afford phosphoric acid dibenzyl ester 2,5-dioxo-4,4-diphenyl-imidazolidin-1-ylmethyl ester of Formula IV; and

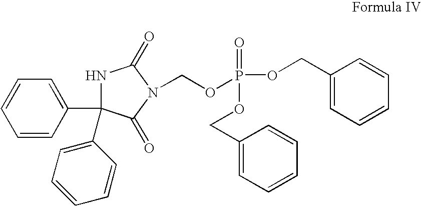

c) reacting dibenzyl ester 2,5-dioxo-4,4-diphenyl-imidazolidin-1-ylmethyl ester of Formula IV with a suitable reagent to afford fosphenytoin of Formula V.

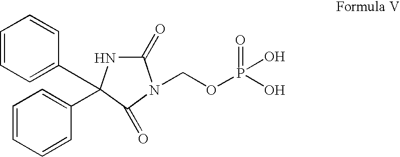

Step a) involves reacting 3-hydroxymethyl-5,5-diphenyl-2,4-imidazolidinedione of Formula II with trichloroacetonitrile in the presence of a suitable base to give 2,2,2-trichloro-acetimidic acid 2,5-dioxo-4,4-diphenyl-imidazolidin-1-yl methyl ester of Formula III.

Suitable solvents which can be used for the reaction include, but are not limited to: ethers such as tetrahydrofuran, 1,4-dioxane and the like; aprotic polar solvents such as DMF, DMSO, DMA and the like, nitriles such as acetonitrile, propionitrile and the like; halogenated hydrocarbons such as dichloromethane, 1,2-dichloroethane, chloroform, carbon tetrachloride and the like; esters such as ethyl acetate, n-propyl acetate, n-butyl acetate, t-butyl acetate and the like; hydrocarbons such as toluene, xylene, n-heptane, cyclohexane, n-hexane and the like; and mixtures thereof.

Suitable bases which can be used include, but are not limited to: organic bases like 1,8-diazabicycl[5.4.0]undec-7-ene, 1,5-diazabicyclo[4.3.0]non-5-ene, triethylamine, and diisopropyl ethyl amine; inorganic bases including alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof.

The amount of base used affects this reaction, as employment of excess base can lead to the formation of 2,2,2-trichloro-N-(2,5-dioxo-4,4-diphenyl-imidazolidin-1-yl-methyl)-acetamide of Formula IIIa as an impurity in the product.

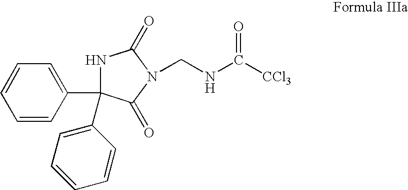

Suitably, the mole ratio of base used for the reaction is less than about 0.03 moles, per mole of 3-hydroxymethyl-5,5-diphenyl-2,4-imidazolidinedione of Formula II. Generally, at least about 0.01 moles of base will be used, per mole of the compound of Formula II.

Suitable temperatures for conducting the reaction range from about 0° C. to about 50° C., or from about 10° C. to about 30° C.

In an embodiment, the product 2,2,2-trichloro-acetimidic acid 2,5-dioxo-4,4-diphenyl-imidazolidin-1-ylmethyl ester of Formula III formed in the reaction medium is progressed to step b) without isolating the compound, thus giving rise to an in-situ process.

Step b) involves reacting 2,2,2-trichloro-acetimidic acid 2,5-dioxo-4,4-diphenyl-imidazolidin-1-ylmethyl ester of Formula III with dibenzyl phosphate to afford phosphoric acid dibenzyl ester 2,5-dioxo-4,4-diphenyl-imidazolidin-1-ylmethyl ester of Formula IV.

Suitable solvents which can be used include, but are not limited to: nitriles such as acetonitrile, propionitrile and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane; halogenated hydrocarbons such as dichloromethane, 1,2-dichloroethane, chloroform, carbon tetrachloride; aprotic polar solvents such as DMF, DMSO, DMA and the like and the like; and mixtures thereof.

Suitable temperatures for conducting the reaction range from about 0° C. to about 100° C., or from about 20° C. to about 60° C.

The reaction can be conducted optionally in the presence of a suitable Lewis or Brönsted acid like boron trifluoride diethyl ether, aluminium chloride, boron trifluoride, iron III chloride, and the like.

The product obtained can optionally be further purified by recrystallization, slurrying, or a combination thereof, in a suitable solvent.

Suitable solvents which can be used for purification include, but are not limited to: alcohols like methanol, ethanol, isopropyl alcohol, n-propanol, and the like; halogenated hydrocarbons such as dichloromethane, 1,2-dichloroethane, chloroform, carbon tetrachloride and the like; esters such as ethyl acetate, n-propyl acetate, n-butyl acetate, t-butyl acetate and the like; hydrocarbons such as toluene, xylene, n-heptane, cyclohexane, n-hexane and the like; and mixtures thereof.

Isolation of the compound during recrystallization may be enhanced by methods such as cooling, partial removal of the solvent from the mixture or by adding an anti-solvent to the reaction mixture, or a combination thereof.

Step c) involves reacting dibenzyl ester 2,5-dioxo-4,4-diphenyl-imidazolidin-1-ylmethyl ester of Formula IV with a suitable reagent, to afford fosphenytoin of Formula V.

Suitable solvents which can be used include, but are not limited to: alcohols such as methanol, ethanol, isopropyl alcohol, n-propanol, and the like; ketones such as acetone, ethyl methyl ketone, methyl isobutyl ketone and the like; esters such as ethyl acetate, n-propyl acetate, n-butyl acetate, t-butyl acetate and the like; hydrocarbons such as toluene, xylene, n-heptane, cyclohexane, n-hexane and the like; nitriles such as acetonitrile, propionitrile and the like; and mixtures thereof.

Suitable reagents for debenzylation include reducing agents, or an acid or a base can be used.

Suitable reducing agents which can be used include a combination of hydrogen and a metal catalyst. Useful metal catalysts include palladium absorbed on carbon, platinum, Raney nickel, platinum dioxide, Raney nickel, and the like. A hydrogen pressure about 1 to about 5 atmospheres, or about 2 to about 5 atomospheres, frequently will be applied for the reaction.

Suitable acids and bases which can be used include, acids like hydrochloric acid, hydrobromic acid, acetic acid, formic acid, sulfuric acid, and the like, and bases like lithium hydride, lithium hydroxide, sodium hydroxide, potassium hydroxide, sodium carbonate, potassium carbonate and the like.

Fosphenytoin formed in the reaction medium can be converted to a base addition salt, optionally without isolating the compound, by reacting it with the desired base in the presence of a suitable solvent.

Suitable bases which can be used to form the base addition salts of fosphenytoin include, but are not limited to: alkali metal hydroxides such as lithium hydroxide, sodium hydroxide, potassium hydroxide and the like; carbonates of alkali metals such as sodium carbonate, potassium carbonate and the like; and bicarbonates of alkali metals such as sodium bicarbonate, potassium bicarbonate, and the like. These bases can be used in the form of solids or in the form of aqueous solutions.

Suitably, aqueous solutions containing about 5% to 50%, or about 10% to 20%, (w/v) of the corresponding base can be used. Any concentration is useful, which will convert the acid addition salt to a free base.

In an embodiment, the base used is sodium hydroxide and the base addition salt of fosphenytoin formed is the fosphenytoin disodium salt of Formula I.

Another aspect of the present invention provides another process for the preparation of fosphenytoin and its pharmaceutically acceptable salts. In an embodiment, the process comprises:

a) reacting 5,5-diphenyl-imidazolidine-2,4-dione of Formula VI with formaldehyde in the presence of a suitable base to afford 3-hydroxymethyl-5,5-diphenyl-imidazolidine-2,4-dione of Formula II;

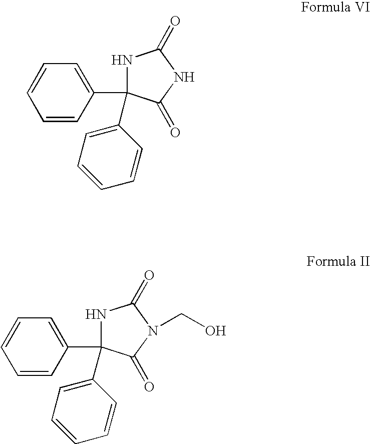

b) reacting 3-hydroxymethyl-5,5-diphenyl-imidazolidine-2,4-dione of Formula II with 3,4-dihydro-2H-pyran of Formula VII in the presence of a suitable base to give 5,5-diphenyl-3-(tetrahydro-pyran-2-yloxymethyl)-imidazolidine-2,4-dione of Formula VIII;

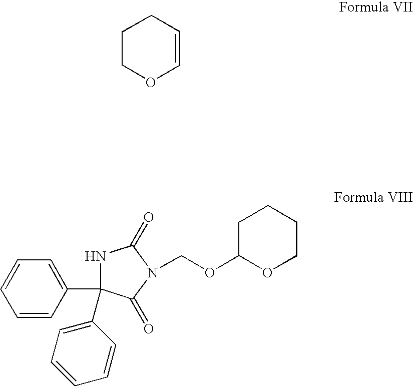

c) reacting 5,5-diphenyl-3-(tetrahydro-pyran-2-yloxymethyl)-imidazolidine-2,4-dione of Formula VIII with a benzyl halide, such as benzyl bromide, in the presence of a suitable base to give 1-benzyl-3-(3,4-dihydro-2H-pyran-2-yloxymethyl)-5,5-diphenyl-imidazolidine-2,4-dione of Formula IX:

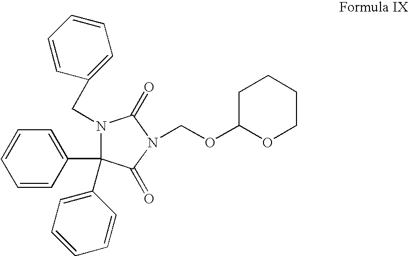

d) reacting 1-benzyl-3-(3,4-dihydro-2H-pyran-2-yloxymethyl)-5,5-diphenyl-imidazolidine-2, 4-dione of Formula IX with a suitable acid to give 1-benzyl-3-hydroxymethyl-5,5-diphenyl-imidazolidine-2,4-dione of Formula X;

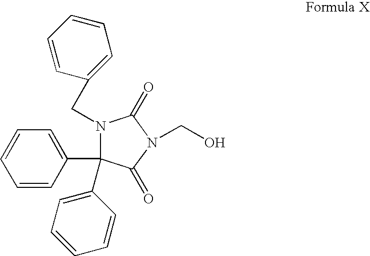

e) reacting 1-benzyl-3-hydroxymethyl-5,5-diphenyl-imidazolidine-2,4-dione of Formula X with a suitable halogenating agent to give 1-benzyl-3-halomethyl-5,5-diphenyl-imidazolidin-3-2,4-dione of Formula XI 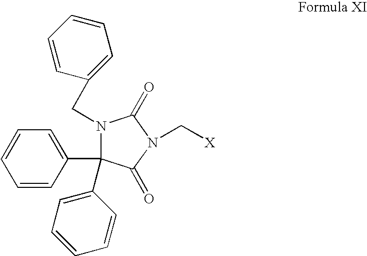
wherein X is Cl, Br, or I;

f) reacting 1-benzyl-3-halomethyl-5,5-diphenyl-imidazolidin-3-2,4-dione of Formula XI with a dibenzyl phosphate in the presence of a suitable base to give phosphoric acid dibenzyl ester 3-benzyl-2,5-dioxo-4,4-diphenyl-imidazolidin-1-yl methyl ester of Formula XII; and

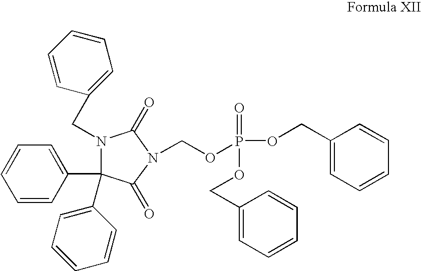

g) reacting phosphoric acid dibenzyl ester 3-benzyl-2,5-dioxo-4,4-diphenyl-imidazolidin-1-ylmethyl ester of Formula XII with a suitable reagent to give fosphenytoin of Formula V.

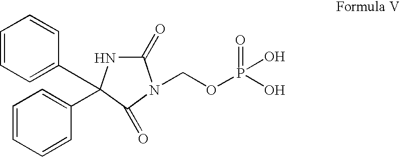

Step a) involves a reaction of 5,5-diphenyl-imidazolidine-2,4-dione of Formula VI with formaldehyde in the presence of a suitable base to afford 3-hydroxymethyl-5,5-diphenyl-imidazolidine-2,4-dione of Formula II.

Suitable solvents which can be used for the reaction include but are not limited to aprotic polar solvents such as DMF, DMSO, DMA and the like, and mixtures thereof or their mixtures with water in various proportions.

Suitable temperatures for conducting the reaction range from about 0 to about 50° C., or from about 10 to about 30° C.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, aqueous solutions containing about 5% to 50%, or about 10% to 20%, (w/v) of the corresponding base can be used. Any concentration is useful, which will convert the acid addition salt to a free base.

Step b) involves a reaction of 3-hydroxymethyl-5,5-diphenyl-imidazolidine-2,4-dione of Formula II with 3,4-dihydro-2H-pyran of Formula VII in the presence of a suitable base to give 5,5-diphenyl-3-(tetrahydro-pyran-2-yloxymethyl)-imidazolidine-2,4-dione of Formula of VIII.

Suitable solvents which can be used for the reaction include, but are not limited to: ethers such as tetrahydrofuran, 1,4-dioxane and the like; aprotic polar solvents such as DMF, DMSO, DMA and the like; and mixtures thereof.

Suitable temperatures for conducting the reaction range from about 0 to about 50° C., or from about 10 to about 30° C.

Suitably, instead of 3,4-dihydro-2H-pyran, other protecting groups such as tetrahydropyran-2-yl, tetrahydrothipyran-2-yl, 4 methoxy tetrahydropyran-2-yl, and tetrahydrofuran-2-yl and the like can be used to give the corresponding protected form of 3-hydroxymethyl-5,5-diphenyl-imidazolidine-2,4-dione of Formula II.

Step c) involves a reaction of 5,5-diphenyl-3-(tetrahydro-pyran-2-yloxymethyl)-imidazolidine-2,4-dione of Formula VIII with benzyl bromide in the presence of a suitable base to give 1-benzyl-3-(3,4-dihydro-2H-pyran-2-yloxymethyl)-5,5-diphenyl-imidazolidine-2,4-dione of Formula IX.

Suitable solvents which can be used for the reaction include, but are not limited to: hydrocarbon solvents such as toluene, xylene, n-heptane, n-hexane, cyclohexane, methylcyclohexane and the like; ethers such as tetrahydrofuran, 1,4-dioxane and the like; aprotic polar solvents such as DMF, DMSO, DMA and the like; and mixtures thereof.

Suitable temperatures for conducting the reaction range from about 0° C. to about 50° C., or from about 20° C. to about 35° C.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, instead of benzyl bromide, other protecting group-forming compounds such as benzyl chloride, benzoyl chloride, benzoyl bromide and the like can be used for protecting the amide group to give the corresponding protected 5,5-diphenyl-3-(tetrahydro-pyran-2-yloxymethyl)-imidazolidine-2,4-dione of Formula VIII.

Step d) involves the hydrolysis of the 1-benzyl-3-(3,4-dihydro-2H-pyran-2-yloxymethyl)-5, 5-diphenyl-imidazolidine-2,4-dione of Formula IX in the presence of suitable acids to give 1-benzyl-3-hydroxymethyl-5,5-diphenyl-imidazolidine-2,4-dione of Formula X.

Suitable acids which can be used for the hydrolysis reaction include, but are not limited to: inorganic acids such as hydrochloric acid, hydrobromic acid, and the like; and organic acids such as acetic acid, formic acid, p-toluene sulfonic acid, and the like.

Suitable temperatures for conducting the reaction range from about 0 to about 50° C., or from about 20 to about 35° C.

Suitable organic solvents which can be used for conducting the reaction include, but are not limited to: alcohols such as methanol, ethanol, isopropyl alcohol, n-propanol, and the like; ketones such as acetone, ethyl methyl ketone, methyl isobutyl ketone and the like; esters such as ethyl acetate, n-propyl acetate, n-butyl acetate, t-butyl acetate and the like; and mixtures thereof.

Step e) involves reaction of 1-benzyl-3-hydroxymethyl-5,5-diphenyl-imidazolidine-2,4-dione of Formula X with a suitable halogenating agent to give 1-benzyl-3-halomethyl-5,5-diphenyl-imidazolidin-3-2,4-dione of Formula XI.

Suitable halogenating agents which can be used include, but are not limited to pivaloyl chloride, thionyl chloride, phosphorus oxychloride, oxalyl chloride, phosphorus trichloride, phosphorus pentachloride, bromine, hydrogen bromide, methyl iodide and the like.

Suitable organic solvents which can be used in the above step include, but are not limited to: halogenated solvents such as dichloromethane, ethylene dichloride and the like; hydrocarbons such as toluene and the like; esters such as ethyl acetate, n-propyl acetate, n-butyl acetate, t-butyl acetate and the like; ethers such as diethyl ether, dimethyl ether, diisopropyl ether, 1,4-dioxane and the like; and mixtures thereof.

Suitable temperatures for conducting the reaction range from about 20° C. to 70° C., or from about 40° C. to about 60° C.

Step f) involves condensation of the 1-benzyl-3-halomethyl-5,5-diphenyl-imidazolidin-3-2,4-dione of Formula XI with a dibenzyl phosphate in the presence of a suitable base to give phosphoric acid dibenzyl ester 3-benzyl-2,5-dioxo-4,4-diphenyl-imidazolidin-1-ylmethyl ester of Formula XII.

Suitable organic solvents which can be used for the reaction include, but are not limited to: ethers such as diethyl ether, dimethyl ether, diisopropyl ether, 1,4-dioxane and the like; hydrocarbons such as toluene, xylene, n-heptane, cyclohexane, n-hexane and the like; nitriles such as acetonitrile, propionitrile and the like; and mixtures thereof.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

The pH of the reaction mass may range from about 7 to about 14, or from about 8 to about 10.

Suitable temperatures for conducting the reaction can range from about 50° C. to about 100° C., or from about 60° C. to about 100° C.

Step g) involves reaction of the phosphoric acid dibenzyl ester 3-benzyl-2,5-dioxo-4, 4-diphenyl-imidazolidin-1-ylmethyl ester of Formula XII with a suitable reducing agent to give fosphenytoin of Formula V.

Suitable solvents which can be used include, but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane and the like; hydrocarbons such as toluene, xylene, n-heptane, cyclohexane, n-hexane and the like; nitriles such as acetonitrile, propionitrile and the like; and mixtures thereof.

Debenzylation can be carried out using reducing agents, or by treatment with an acid or a base.

Suitable reducing agents which can be used include a combination of hydrogen and a metal catalyst. Useful metal catalysts include palladium absorbed on carbon, platinum, Raney nickel, platinum dioxide, Raney nickel, and the like. A hydrogen pressure about 1 to about 5 atmospheres, or about 2 to about 5 atomospheres, frequently will be applied for the reaction.

Acidic or alkaline conditions can be provided using any acid like hydrochloric acid, hydrobromic acid, acetic acid, formic acid, sulfuric acid, and the like, or any base like lithium hydride, lithium hydroxide, sodium hydroxide, potassium hydroxide, sodium carbonate, potassium carbonate, and the like.

Fosphenytoin formed in the reaction medium can be converted to its base addition salts, optionally without isolating the compound, by reacting it with the desired base in the presence of a suitable solvent.

Suitable bases which can be used to form the base addition salts of fosphenytoin include, but are not limited to: alkali metal hydroxides such as lithium hydroxide, sodium hydroxide, potassium hydroxide and the like; carbonates of alkali metals such as sodium carbonate, potassium carbonate and the like; and bicarbonates of alkali metals such as sodium bicarbonate, potassium bicarbonate, and the like. These bases can be used in the form of solids or in the form of aqueous solutions.

Suitably, aqueous solutions containing about 5% to 50%, or about 10% to 20%, (w/v) of the corresponding base can be used. Any concentration is useful, which will convert the acid addition salt to a free base.

In an embodiment, the base used is sodium hydroxide and the base addition salt of fosphenytoin formed is the fosphenytoin disodium salt of Formula I.

Yet another aspect of the present invention provides a process for the conversion of fosphenytoin to its sodium salt using about 2 to less than about 2.5 moles of sodium hydroxide, per mole of fosphenytoin.

Fosphenytoin can be converted to its disodium salt by reacting it with sodium hydroxide in the presence of a suitable solvent.

Suitable solvents include, but are not limited to: alcohols such as methanol, ethanol, isopropyl alcohol, n-propanol, and the like; ketones such as acetone, ethyl methyl ketone, methyl isobutyl ketone and the like; and mixtures thereof, or their mixtures with water in various proportions.

Suitably, aqueous solutions containing about 5% to 50%, or about 10% to 20%, (w/v) of sodium hydroxide can be used.

The mole ratio of sodium hydroxide used is important since an excess mole ratio of sodium hydroxide can lead to the formation of disodium hydrogen phosphate (hereinafter referred to as a “phosphate salt”) as an impurity in the final product.

The phosphate salt impurity content in fosphenytoin sodium can be determined using a high performance liquid chromatography (“HPLC”) method.

The amount of sodium hydroxide can range from about 2 to about 2.5 moles, per mole of fosphenytoin. Use of less than about 2 moles of sodium hydroxide, per mole of fosphenytoin, provides a lower yield of product due to a lessened conversion into the salt.

The fosphenytoin sodium formed in the reaction medium can be isolated using techniques such as crystallization. Suitably, crystallization is carried out by adding an excess of a ketone solvent to the solution comprising fosphenytoin sodium.

The solid product thus obtained can be further purified by using recrystallization or slurrying.

For recrystallization, a solution can be prepared at an elevated temperature if desired to achieve a desired concentration. Any temperature is acceptable for the dissolution as long as a clear solution of fosphenytoin disodium salt is obtained. The solution may be brought down to room temperature for further processing if required or an elevated temperature may be used. A higher temperature will allow the precipitation from solutions with higher concentrations of fosphenytoin sodium, resulting in better economy of manufacture.

For crystallization to occur, the reaction mass may be maintained further at temperatures lower than the concentration temperatures such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry.

Suitable solvents which can be used for recrystallization or slurrying include, but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; and mixtures thereof, or their mixtures with water in various proportions.

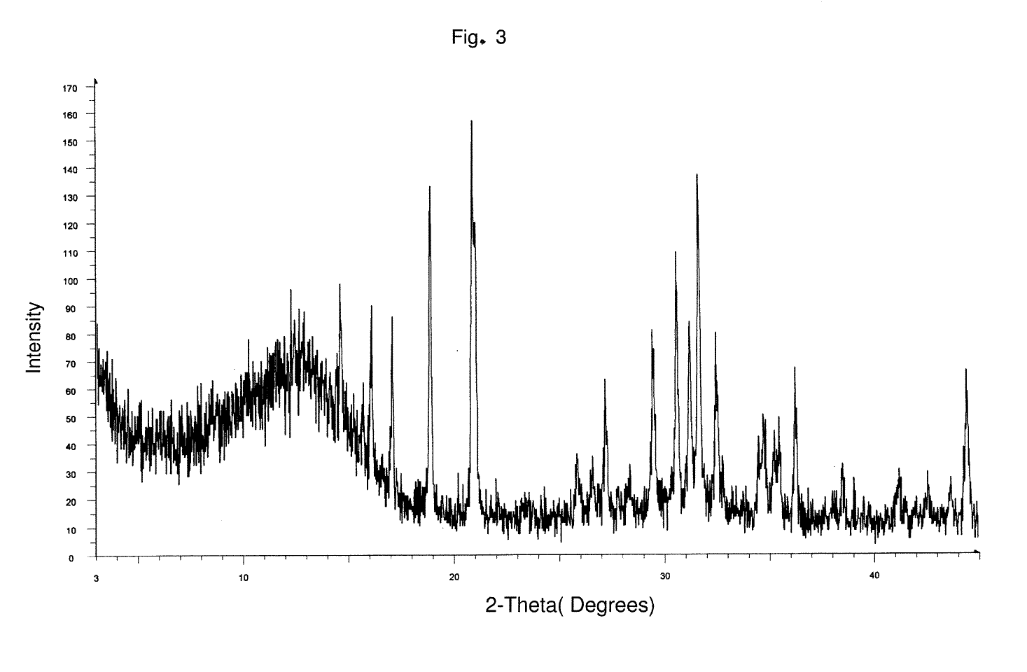

Fosphenytoin sodium obtained above is in the form of a crystalline solid characterized by an X-ray powder diffraction (“XRPD”) pattern substantially in accordance with the pattern of FIG. 3. XRPD data were obtained using Cu Kα radiation, having the wavelength 1.541 Å and were obtained using a Bruker AXS D8 Advance Powder X-ray Diffractometer. Crystalline fosphenytoin sodium obtained is also characterized by an XRPD pattern having significant peaks at about 14.6, 16.0, 17.0, 18.9, 20.9, 27.2, 29.5, and 30.6, ±0.2 degrees 2θ. The pattern is also characterized by additional XRPD peaks at about 31.2, 31.6, 32.5, and 36.3, ±0.2 degrees 2θ.

Still another aspect of the present invention provides the following intermediates that are useful for the preparation of fosphenytoin and its pharmaceutically acceptable salts:

a) 2,2,2-trichloro-acetimidic acid 2,5-dioxo-4,4-diphenyl-imidazolidin-1-ylmethyl ester of Formula III;

b) 5 ,5-diphenyl-3-(tetrahydro-pyran-2-yloxymethyl)-imidazolidine-2,4-dione of Formula VIII;

c) 1-benzyl-5,5-diphenyl-3-(tetrahydro-pyran-2-yloxymethyl)-imidazolidine-2,4-dione of Formula IX;

d) 1-benzyl-3-hydroxymethyl-5,5-diphenyl-imidazolidine-2,4-dione of Formula X;

e) 1-benzyl-3-halomethyl-5,5-diphenyl-imidazolidine-2,4-dione of Formula XI; and

f) phosphoric acid dibenzyl ester 3-benzyl-2,5-dioxo-4,4-diphenyl-imidazolidin-1-yl methyl ester of Formula XII.

Still another aspect of the present invention provides substantially pure fosphenytoin and its pharmaceutically acceptable salts.

Fosphenytoin and its salts obtained according to the process of the present invention are substantially pure. By “substantially pure fosphenytoin” it is meant that fosphenytoin and its salts prepared in accordance with the present invention contain less than about 0.5%, or less than about 0.15%, by weight of each of the corresponding impurities like the diphenylglycine, diphenylhydantoic acid, 3-hydroxymethyl-5,5-diphenyl-2,4-imidazolidinedione of Formula II, 5,5-diphenyl-imidazolidine-2,4-dione of Formula VI, phosphoric acid dibenzyl ester 2,5-dioxo-4,4-diphenyl-imidazolidin-1-ylmethyl ester of Formula IV, and the amide impurity 2,2,2-trichloror-N-(2,5-dioxo-4,4-diphenyl-imidazolidin-1-yl-methyl)-acetamide of Formula IIIa, as characterized by a HPLC chromatogram obtained from a mixture comprising the desired compound and one or more of the said impurities.

Fosphenytoin prepared according the process of the present invention has an assay by HPLC ranging from about 98 to about 102%, or from about 99 to about 101%, by weight. The content of the phosphate salt impurity will be less than about 2 percent by weight.

Fosphenytoin prepared according to the process of the present invention contains less than about 5000 ppm, or less than about 3000 ppm, or less than about 1000 ppm, of individual residual organic solvents as determined using techniques such as gas chromatography (“GC”).

Fosphenytoin prepared according to the process of the present invention typically contains less than about 100 ppm of each of n-heptane, acetone, ethyl acetate, and methanol, less than about 35 ppm of isopropyl alcohol, less than about 75 ppm of acetonitrile, and less than about 185 ppm of toluene.

Still another aspect of the present invention provides amorphous fosphenytoin sodium and a process for its preparation.

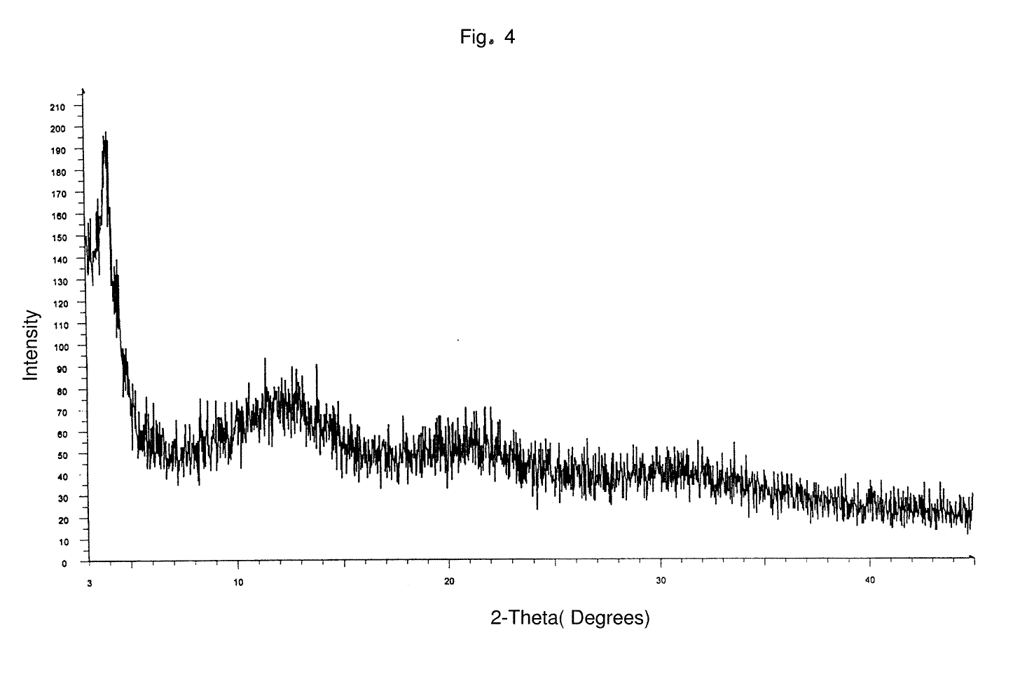

Amorphous fosphenytoin sodium is characterized by its XRPD pattern. The amorphous form of fosphenytoin sodium is characterized by an XRPD pattern showing a plain halo with no peaks, which is characteristic of an amorphous solid, substantially in accordance with the pattern of FIG. 4.

An embodiment of a process for the preparation of amorphous fosphenytoin sodium involves recrystallization of fosphenytoin sodium in acetone. Recrystallization involves providing a solution of fosphenytoin sodium in acetone and then crystallizing the solid from the solution.

The concentration of fosphenytoin sodium in the solvent can range from 40 to 80% or more. The solution can be prepared at an elevated temperature if desired to achieve a higher solute concentration. Any temperature is acceptable for the dissolution as long as a clear solution of fosphenytoin sodium is obtained and is not detrimental to the drug substance chemically or physically. The solution may be brought down to a lower temperature for further processing if required or an elevated temperature may be used. A higher temperature for dissolution will allow the precipitation from solutions with higher concentrations of fosphenytoin sodium, resulting in better economy of manufacture.

The precipitated product may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods to achieve the desired product purity, times from about 1 to 20 hours, or longer, frequently being adequate.

Still more another aspect of the present invention provides a pharmaceutical composition comprising fosphenytoin or a pharmaceutically acceptable salt, prepared according to the process of the present invention, together with one or more pharmaceutically acceptable excipients.

Fosphenytoin sodium prepared according to the process of the present invention is well suited for use in preparing pharmaceutical formulations.

The pharmaceutical formulations according to the present invention include liquid oral dosage forms such as solutions, dispersions, suspensions, emulsions and so on; parenteral dosage forms (including intramuscular, subcutaneous, intravenous) such as injectable dosages by solution or suspension or dispersions or sterile powders for reconstitution; transdermally delivery systems; targeted delivery systems etc. For liquid oral dosage forms, the compositions include but not limited to pharmaceutically acceptable aqueous or non aqueous vehicles etc., flavoring agents, preservatives, solubilizers, emulsifiers, etc.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations may be adapted for topical administration including but not limited to ointments, creams, suspensions, lotions, powders, solutions, pastes, gels, sprays, aerosols or oils.

The pharmaceutical formulations can further be packed in vials or ampoules made of glass, containers and lids of high-density polyethylene (HDPE), low-density polyethylene (LDPE) and or polypropylene and/or glass, and blisters or strips composed of aluminium or high-density polypropylene. These lists are not intended to be exhaustive, as other materials and packaging types are also useful.

In the compositions of present invention, fosphenytoin or its salt is a useful active ingredient in the range of 50 mg to 100 mg, or 70 mg to 80 mg, in a dosage unit.

Certain specific aspects and embodiments of this invention are described in further detail by the examples below, which examples are provided only for the purpose of illustration and are not intended to limit the scope of the appended claims in any manner.

### EXAMPLE 1

### PREPARATION OF 5,5-DIPHENYL-3-(TETRAHYDRO-PYRAN-2-YLOXYMETHYL)-IMIDAZOLIDINE-2,4-DIONE OF FORMULA II

390 liters of water was taken into a reactor and 0.64 kg of potassium carbonate was added. The mixture was stirred at 33° C. for 15 minutes to get a clear solution, and 14.7 kg of 40% aqueous formaldehyde was added and then stirred for 15 minutes. 13 kg of 5,5-diphenyl-2, 4-imidazolidinedione (phenytoin) was added to the solution followed by addition of 78 liters of water. The reaction mass was maintained at 30° C. for 2 hours, and reaction completion was checked using HPLC. After the reaction was completed, the reaction mass was passed through a centrifuge and the solid was washed with 52 liters of water, followed by washing with 78 liters of water in two equal lots. The wet material was dried at 57° C. for 6 hours to yield 13.5 kg of the title compound.

### EXAMPLE 2

### PREPARATION OF 2,2,2-TRICHLORO-ACETIMIDIC ACID 2,5-DIOXO-4,4-DIPHENYL-IMIDAZOLIDIN-1-YLMETHYL ESTER (FORMULA III)

30 g of 3-hydroxymethyl-5,5-diphenyl-imidazolidine-2,4-dione of Formula II was placed into a clean and dry round bottom flask containing 300 ml of tetrahydrofuran and stirred at 27° C. for 10 minutes. 14.68 g of potassium carbonate and 12 ml of trichloroacetonitrile of Formula V were added slowly to the reaction mass and stirred at 27° C. for 4 hours. Reaction progress was checked using thin layer chromatography. After the reaction was complete, the reaction mass was filtered and the filtrate was distilled completely at 50° C. under a vacuum of 650 mm Hg to afford 37 g of the title compound.

Mass (m/z)=427.8 a.m.u.

### EXAMPLE 3

### PREPARATION OF PHOSPHORIC ACID DIBENZYL ESTER 2,5-DIOXO-4,4-DIPHENYL-IMIDAZOLIDIN-1-YLMETHYL ESTER (FORMULA IV)

108 liters of acetonitrile was taken into a reactor and 12.0 kg of 3-hydroxymethyl-5,5-diphenyl-2,4-imidazolidinedione of Formula II was added to it. The reaction mass was maintained at 28° C. for 20 minutes. 9.91 kg of trichloroacetonitrile was added to the reaction mass and maintained at 28° C. for 20 minutes. A solution of 0.162 kg of 1,8-diazabicyclo[5.4.0]undec-7-ene in 12 liters of acetonitrile was prepared and added into the reaction mass slowly at 30 to 32° C. and maintained at the same temperature for 4 hours. Reaction progress was monitored using thin layer chromatography. After the reaction was complete, a solution of 9.47 kg of dibenzyl phosphate in 132 liters of acetonitrile was added slowly at 30 to 31° C. The reaction mass was stirred at 30° C. for 3 hours, and then the solvent was distilled completely at 59° C. and a vacuum of 700 mm Hg, then the residue was maintained at 59° C. for 20 minutes. 36 liters of ethyl acetate was added to the residue and stirred at 30° C. for 15 minutes. The solvent was distilled completely at 54° C. and a vacuum of 700 mm Hg and the residue was maintained under the same conditions for a further 20 minutes. The reaction mass was then cooled to 30° C. and 120 liters of ethyl acetate was added and maintained at the same temperature under stirring for 15 minutes. Another 120 liters of ethyl acetate was added and maintained under stirring for another 15 minutes. The organic layer was checked for clear dissolution and then washed with 360 liters of water in three equal lots. The organic layer was treated with carbon, filtered, and the carbon bed was washed with 30 liters of ethyl acetate. The combined organic layer was distilled completely at 50° C. under a vacuum of 700 mm Hg. The residue obtained was co-distilled with 36 liters of isopropyl alcohol and then 24 liters of isopropyl alcohol was added to the residue and cooled to 30° C. The mass was maintained at 30° C. for 10 minutes to form a clear solution of the title compound.

24 liters of n-heptane was taken into another reactor and cooled to 25° C. 0.048 kg of the title compound was added to it as seed for crystallization. The solution of the compound in isopropyl alcohol was added to the cooled heptane slowly at 25° C. The mass was maintained at 25° C. for 3 hours. 24 liters of n-heptane was added to the mass slowly in 4 equal lots at uniform time intervals by maintaining for 3 hours after the addition of each lot. The separated solid was filtered and washed with a mixture of 24 liters of isopropyl alcohol and 3 liters of n-heptane.

12 liters of isopropyl alcohol was taken into another reactor and cooled to 25° C. The separated solid above was added and 12 liters of heptane was added slowly. The mass was maintained at 28° C. for 20 minutes and then filtered and washed with 24 liters of a 1:1 mixture of isopropyl alcohol and n-heptane. The wet material was dried at 52° C. and a vacuum of 650 mm Hg for 5 hours to yield 9.0 kg of the title compound.

### EXAMPLE 4

### PREPARATION OF PHOSPHORIC ACID DIBENZYL ESTER 2,5-DIOXO-4,4-DIPHENYL-IMIDAZOLIDIN-1-YLMETHYL ESTER (FORMULA IV)

23 g of dibenzyl phosphate was taken into a clean and dry round bottom flask containing 115 ml of acetonitrile and the mixture was stirred for 10 minutes at 28° C. The mixture was then cooled to 3° C. and 3.0 ml of a 45% solution of boron trifluoride in diethyl ether was added slowly under a nitrogen atmosphere. The resultant mixture was stirred for 15 minutes, and then a solution of 35.2 g of 2,2,2-trichloro-acetimidic acid 2,5-dioxo-4,4-diphenyl-imidazolidin-1-ylmethyl ester of Formula VII in 176 ml of dichloromethane was added slowly at 3° C. The resultant mixture was stirred at 28° C. for 15 minutes followed by addition of 460 ml of water and 460 ml of dichloromethane. The organic layer was separated and the aqueous layer was extracted with 230 ml of dichloromethane. The combined organic layer was washed with 2×230 ml of water. The organic layer was distilled completely at 50° C. under a vacuum of 650 mm Hg to afford 35 g of the title compound as a syrup.

Mass (m/z)=543 a.m.u.

### EXAMPLE 5

### PREPARATION OF FOSPHENYTOIN DISODIUM SALT (FORMULA I)

119 liters of methanol and 8.5 kg of phosphoric acid dibenzyl ester 2,5-dioxo-4,4-diphenyl-imidazolidin-1-ylmethyl ester of Formula IV were taken into a reactor and stirred at 29° C. for 20 minutes. A solution of 0.85 kg of wet 5% palladium on charcoal in 26 liters of methanol was prepared and added to the above reaction mass. Another 8.5 liters of methanol was added to the same reaction mass and maintained under a nitrogen atmosphere for 20 minutes at 25° C. A hydrogen pressure of 3 to 3.5 Kg/cm² was applied to the reaction mass and maintained at 35° C. for 5 hours. Reaction progress was monitored using thin layer chromatography. After the reaction was complete, the reaction mass was filtered through a celite bed and the bed was washed with 34 liters of methanol. The filtrate was distilled completely at 7 to 10° C. under a vacuum of 700 mm Hg. A mixture of 34 liters of acetone and 3.4 liters of water was added to the residue obtained, cooled to 30° C. and maintained for 20 minutes. A solution of 1.25 kg of sodium hydroxide flakes in 3.75 liters of water was added to the reaction mass slowly at 5° C. and maintained at the same temperature for 45 minutes. 90 liters of acetone was added to the above reaction mass slowly and maintained at 3 to 4° C. for 3.5 hours. The solid that separated was filtered and the solid was washed with 8.5 liters of acetone. The wet material obtained was stirred in 34 liters of chilled acetone for 30 minutes and then filtered and washed with 8.5 liters of acetone. A mixture of 26 liters of methanol and 0.85 liters of water was prepared and cooled to 3° C. The wet material was added to the cooled solution and maintained for 30 minutes. The solid was filtered and washed with 8.7 liters of methanol. The wet solid was dried at 33° C. under a vacuum of 650 mm Hg for 6 hours to yield 5.5 kg of the title compound.

### EXAMPLE 6

### PREPARATION OF FOSPHENYTOIN SODIUM (FORMULA I)

35.1 liters of ultra-filtered water and 3.9 kg of fosphenytoin sodium prepared in Example 5 were taken into a reactor and stirred at 25° C. for 20 minutes. The reaction was checked for formation of a clear solution and then pH of the solution was adjusted to 9.1 with a solution of 0.195 liters of 35% hydrochloric acid in 8.5 liters of water. The solution was treated with carbon, filtered, and the carbon bed was washed with 7.8 liters of water. 166 liters of acetone was taken into a separate reactor and the filtrate was added to it and stirred for 2 hours at 25° C., then was further cooled to 4° C. and maintained for 4 hours. The separated solid was filtered and washed with 7.8 liters of chilled acetone. The wet material was dried at 32° C. and a vacuum of 700 mm Hg for 10 hours to yield 2.6 kg of the title compound. Finally the material was milled in a multi-mill and sieved through a 20 mesh sieve.

Moisture Content: 25.3%.

Purity by HPLC: 99.97%

Individual impurities by HPLC: Less than 0.05%.

Assay by HPLC: 100.3%.

Residual organic solvents: acetone: 259 ppm; methanol: 85 ppm; other individual organic solvents: less than 10 ppm.

### EXAMPLE 7

### PREPARATION OF FOSPHENYTOIN SODIUM (FORMULA I)

35 g of phosphoric acid dibenzyl ester 3-benzyl-2,5-dioxo-4,4-diphenyl-imidazolidin-1-yl methyl ester of Formula IV was taken into a clean and dry autoclave containing 350 ml of ethyl acetate and 18 g of wet 10% palladium on carbon was added. The resultant mixture was maintained at 30° C. under anhydrous hydrogen pressure of 3-4 kg/cm² for 3 hours. Reaction progress was monitored using thin layer chromatography. After the reaction was complete, the reaction mixture was filtered through a celite bed followed by washing the celite with 100 ml of ethyl acetate. The resultant filtrate was distilled completely at 30° C. under a vacuum of 650 mm Hg to afford a crude free base of Formula I with Mass m/z=362 a.m.u.

The above-obtained residue was dissolved in 140 ml of methanol and the solution was treated with carbon, then was then filtered through a celite bed and the bed was washed with 35 ml of methanol. To the filtrate, 4 g of sodium hydroxide was added followed by cooling to 5° C. The mixture was stirred at about 5° C. for 40 minutes. The solid that separated was filtered and washed with 35 ml of methanol. The solid obtained was dried at 50° C. under a vacuum of 650 mm Hg for 8 hours to afford 14.8 g of the title compound, having the X-ray powder diffraction pattern of FIG. 3.

Mass (m/z)=428 a.m.u.

### EXAMPLE 8

### PREPARATION OF 3-HYDROXYMETHYL-5,5-DIPHENYL-IMIDAZOLIDINE-2,4-DIONE (FORMULA VII)

360 ml of water was taken into a clean and dry round bottom flask containing 0.5 g of potassium carbonate. The mixture was stirred for 20 minutes. 10 g of 5,5-diphenyl-2,4-imidazolidinedione of Formula VI and 40 ml of formaldehyde were added to the above solution and the resulting mixture was heated to 57° C. The reaction mass was maintained at 57-58° C. for 1 hour, and then cooled to 30° C. The separated solid was filtered and washed with 300 ml of water followed by drying the solid obtained at 45° C. under a vacuum of 400 mm Hg for 7 hours to afford 10.1 g of the title compound.

Mass (m/z)=282 a.m.u.

### EXAMPLE 9

### PREPARATION OF 5,5-DIPHENYL-3-(TETRAHYDRO-PYRAN-2-YLOXYMETHYL)-IMIDAZOLIDINE-2,4-DIONE (FORMULA VIII)

15 g of 3-hydroxymethyl-5,5-diphenyl-imidazolidine-2, 4-dione of Formula VII was taken into a clean and dry round bottom flask containing 60 ml of tetrahydrofuran. The reaction mixture was stirred for 10 minutes. 7.1 ml of 3,4-dihydro-2H-pyran and 0.4 g of acidified silica gel were added and the resulting reaction mixture was heated to 37° C. for 4 hours. After the reaction was complete, 100 ml of water and 100 ml of ethyl acetate were added and stirred for 10 minutes. The organic layer was separated and distilled completely under a vacuum of 350 mm Hg and a temperature of 50° C. to afford 35 g of the title compound as a residue. The residue was purified by column chromatography using petroleum ether and ethyl acetate in the ratio of 8.6:1.4 as the eluent to afford 2.5 g of pure title compound.

Mass (m/z)=366 a.m.u.

### EXAMPLE 10

### PREPARATION OF 1-BENZYL-5,5-DIPHENYL-3-(TETRAHYDRO-PYRAN-2-YLOXYMETHYL)-IMIDAZOLIDINE-2,4-DIONE (FORMULA IX)

0.013 g of sodium hydride was taken into a clean and dry round bottom flask containing 5 ml of N,N-dimethylformamide. The mixture was stirred for 20 minutes and then 0.1 g of 5,5-diphenyl-3-(tetrahydro-pyran-2-yloxymethyl)-imidazolidine-2,4-dione of Formula VIII was added. The reaction mixture was maintained at 30° C. for 15 minutes, then 0.042 ml of benzyl bromide of Formula VII was added at 30° C. and maintained for 30 minutes. Reaction progress was monitored using thin layer chromatography. After completion of the reaction, 5 g of ice and 30 ml of ethyl acetate was added to the reaction mass and stirred for about 10-15 minutes followed by separation of organic and aqueous layers. The organic layer was washed with water. The organic and aqueous layers were separated and the organic layer was dried using sodium sulphate. The organic layer was distilled completely under vacuum to afford 0.08 g of title compound as a residue.

Mass (m/z)=479 a.m.u. (as a sodium adduct).

### EXAMPLE 11

### PREPARATION OF 1-BENZYL-3-HYDROXYMETHYL-5,5-DIPHENYL-IMIDAZOLIDINE-2,4-DIONE (FORMULA X)

11 g of 1-benzyl-5,5-diphenyl-3-(tetrahydro-pyran-2-yloxymethyl)-imidazolidine-2,4-dione of Formula IX of was charged into a clean and dry 4 neck round bottom flask containing 50 ml of methanol. The mixture was stirred for 20 minutes. 5 ml of hydrochloric acid was added and the resulting reaction mixture was maintained at 25-30° C. for 40 minutes. After completion of the reaction, 100 ml of ethyl acetate was charged and stirred for 15 minutes followed by separation of organic and aqueous layers. The organic layer was distilled under vacuum to afford 9.3 of title compound as a residue.

Mass (m/z)=372 a.m.u.

### EXAMPLE 12

### PREPARATION OF 1-BENZYL-3-CHLOROMETHYL-5,5-DIPHENYL-IMIDAZOLIDIN-3-2,4-DIONE (FORMULA XI)

9 g of 1-benzyl-3-hydroxymethyl-5,5-diphenyl-imidazolidine-2,4-dione of Formula X was taken into a clean and dry round bottom flask containing 60 ml of ethyl acetate and 0.2 ml of DMF was added. The mixture was stirred for 20 minutes and then 3.5 ml of thionyl chloride was added. The resulting reaction mixture was heated to 60° C. and maintained for 40 minutes. Reaction progress was monitored using thin layer chromatography. After the reaction was completed, the mass was cooled to 30° C. followed by addition of 200 ml of 10% aqueous sodium bicarbonate solution and stirring for 15 minutes. The organic layer was separated and distilled completely under a vacuum of 300 mm Hg at 30° C. The residue was co-distilled with 100 ml of petroleum ether to remove traces of thionyl chloride and to afford the title compound as a residue. 50 ml of petroleum ether was added to the residue and the resultant suspension was stirred for 30 minutes. The separated solid was filtered and dried at 35° C. under a vacuum of 300 mm Hg for 30 minutes to afford 3.7 g of the title compound in a pure form.

Mass (m/z)=390 a.m.u.

### EXAMPLE 13

### PREPARATION OF PHOSPHORIC ACID DIBENZYL ESTER 3-BENZYL-2,5-DIOXO-4,4-DIPHENYL-IMIDAZOLIDIN-1-YLMETHYL ESTER (FORMULA XII)

1 g of 1-Benzyl-3-chloromethyl-5,5-diphenyl-imidazolidine-2,4-dione of Formula XI was taken into a clean and dry round bottom flask containing 10 ml of acetonitrile. The reaction mixture was stirred for 10 minutes. 0.87 g of sodium dibenzyl phosphate and 0.04 g of potassium bicarbonate was added and the resulting reaction mixture was heated to 90° C. The reaction mass was maintained at 87° C. for 8 hours. Reaction progress was monitored using thin layer chromatography. After the reaction was complete, the reaction mass was cooled to 28° C. and filtered through a celite bed. The celite was washed with 10 ml of acetonitrile. The filtrate was distilled completely under a vacuum of 300 mm Hg to afford 1.4 g of the title compound as a residue.

Mass (m/z)=632 a.m.u. (655 as a sodium adduct).

### EXAMPLE 14

### PREPARATION OF AMORPHOUS FOSPHENYTOIN DISODIUM SALT (FORMULA I)

4.5 g of phosphoric acid dibenzyl ester 3-benzyl-2,5-dioxo-4,4-diphenyl-imidazolidin-1-yl methyl ester of Formula XII was taken in an autoclave containing 45 ml of acetone. 2 g of wet 10% palladium on carbon was added and the resultant reaction mixture was maintained under 2.5-3 kg/cm² hydrogen pressure at 30° C. The reaction mixture was agitated for 45 minutes. After completion of the reaction, the reaction mixture was filtered through a celite bed and the celite was washed with 15 ml of acetone. The filtrate was distilled completely at 30° C. under a vacuum of 350 mm Hg to afford a residue.

The above-obtained residue was dissolved in 40 ml of methanol and 0.5 g of carbon was added to it. The mixture was stirred at 45° C. for 10 minutes and then filtered through a celite bed. The bed was washed with 10 ml of methanol. The pH of the filtrate was adjusted to 8.8 with 3 ml of 30% aqueous sodium hydroxide solution. To the resultant filtrate, 100 ml of acetone was added and stirred for 30 minutes. The separated solid was filtered and washed with 10 ml of acetone. The wet solid was dissolved in 50 ml of acetone and stirred for 45 minutes. The resultant suspension was filtered and washed with 10 ml of acetone. The wet solid was dried at 65° C. under a vacuum of 300 mm Hg for 6 hours to afford 1.6 g of the title compound having the X-ray powder diffraction pattern of FIG. 4.

Mass (m/z)=406 a.m.u.

### EXAMPLE 15

### PREPARATION OF 2,2,2-TRICHLOROR-N-(2,5-DIOXO-4,4-DIPHENYL-IMIDAZOLI-DIN-1-YL-METHYL)-ACETAMIDE (FORMULA IIIA)

5 g of 2,2,2-trichloro-acetimidic acid 2,5-dioxo-4,4-diphenyl-imidazolidin-1-ylmethyl ester of Formula III, 25 ml of dichloromethane and 3.0 g of trichloroacetonitrile were taken into a round bottom flask and stirred at 28° C. for 10 minutes. 2.6 ml of 1,8-diazabicycl[5.4.0]undec-7-ene was added to the above reaction mass and maintained at 28° C. for 35 minutes. The reaction mass was then distilled completely at 55° C. to remove the solvent. To the residue, 15 ml of chloroform was added and cooled to 5° C., and maintained for 1.5 hours. The separated solid was filtered and washed with 5 ml of chloroform. The wet solid was dried at 25° C. for 45 minutes to yield 3.5 g of the title compound.

Mass (m/z)=423.8.

### EXAMPLE 16

### Determination of Impurities in Fosphenytoin Sodium

Determining the level of impurities in fosphenytoin and its salts using HPLC. The HPLC analysis conditions are as described in Table 1.

TABLE 1
HPLC method for detecting the level of the impurities.

| Column: | Symmetry shield RP-18 250 × 4.6 × 5.0 μm | | |
|---|---|---|---|
| | or equivalent. | | |
| Wavelength: | 214 nm. | | |
| Flow Rate | 1.0 ml/minute. | | |
| Column | 35° C. | | |
| temperature: | | | |
| Load | 10 μl | | |
| Diluent: | Buffer and acetonitrile in the ratio of 65:35 | | |
| Concentration | 3 mg/ml | | |
| Buffer: | 6.8 g of potassium dihydrogen phosphate and 30 ml of | | |
| | 0.5 molar dodecyltriethylammonium phosphate | | |
| | dissolved in 1000 ml of water and the pH was adjusted | | |
| | to 3.0 with phosphoric acid. | | |
| Mobile Phase A | 750 ml of buffer and 250 ml of acetonitrile was mixed | | |
| | and degassed through a 0.45 μm membrane filter. | | |
| Mobile Phase B | 250 ml of buffer and 750 ml of acetonitrile was mixed | | |
| | and degassed through a 0.45 μm membrane filter. | | |
| Gradient | Time (in minutes) | % A | % B |
| Program: | 0.01 | 80 | 20 |
| | 2.5 | 80 | 20 |
| | 20.0 | 65 | 35 |
| | 35.0 | 50 | 50 |
| | 50.0 | 50 | 50 |
| | 52.0 | 80 | 20 |
| | 65.0 | 80 | 20 |

| IMPURITY NAME | RRT |
|---|---|
| Diphenylglycine | 0.19 |
| Diphenylhydantoic acid | 0.54 |
| Compound of Formula II | 0.62 |
| Compound of Formula VI | 0.67 |
| Compound of Formula IV | 3.01 |

A relative retention time (“RRT”) is obtained in a chromatogram by dividing the observed retention time for a compound by the observed retention time for fosphenytoin.

### EXAMPLE 17

### Determination of Phosphate Salt Impurity in Fosphenytion Sodium

The level of phosphate salt impurity in fosphenytoin sodium was determined using HPLC. The HPLC analysis conditions are as described in Table 2.

TABLE 2
HPLC method for detecting the level of phosphate salt impurity.

| Column: | Allsep Anion 150 × 4.6 mm, 7 um or equivalent | |
|---|---|---|
| Flow Rate | 0.7 ml/min. | |
| Column | 60° C. | |
| temperature: | | |
| Injection volume: | 60 μl | |
| Diluent: | Mobile phase | |
| Elution: | Isocratic. | |
| Mobile phase: | 0.5 ml formic acid in one liter of water and pH | |
| | adjusted to 4.0 with liquid ammonia. | |
| Run time: | 30 minutes. | |
| Detector | Polarity | Positive |
| Parameters: | Purge time | 2.0 min |
| | Optical unit temperature | 35° C. |
| | Attenuation | 500 × 10³ nRIU |

Peak area is compared with peak areas obtained from a series of standard solutions, to determine the amount of salt in a sample.

### EXAMPLE 18

### Effect of Mole Ratio of Sodium Hydroxide on Phosphate Salt Formation

Fosphenytoin was converted to its sodium salt, following a process similar to that described in Example 5. Experiments were carried out with varying mole ratios of sodium hydroxide, and phosphate salt content was determined in the products using HPLC. The results are tabulated below.

| | Sodium Hydroxide:Fosphenytoin | |
|---|---|---|
| Experiment | Mole Ratio | Phosphate Salt Content |
| 1 | 2.0 | 0.28% |
| 2 | 2.6 | 0.35% |
| 3 | 3.6 | 4.13% |

It is evident from these results that an increase in the mole ratio of sodium hydroxide leads to an increase in the percentage of phosphate salt in the product.

1. A process for preparing fosphenytoin or a salt thereof, comprising reacting a: compound having a formula: 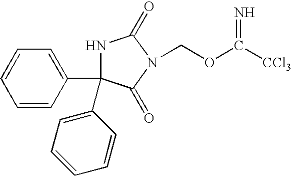
with dibenzyl phosphate, to from a compound having a formula:

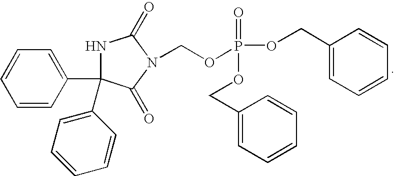

2. The process of claim 1, wherein a compound having a formula: 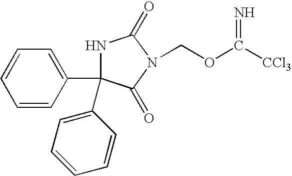
is prepared by reacting a compound having a formula: 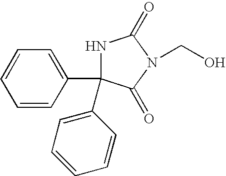
with trichloroacetonitrile, in the presence of a base.

3. The process of claim 2, wherein a compound having a formula: 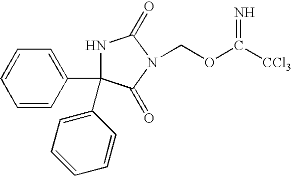
is not isolated, prior to reacting with dibenzyl phosphate.

4. The process of claim 1, further comprising reducing a compound having a formula: 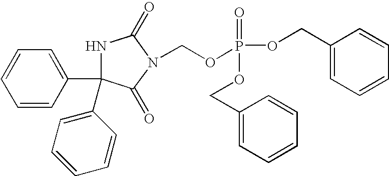
with hydrogen, to form fosphenytoin.

5. A compound having a formula:

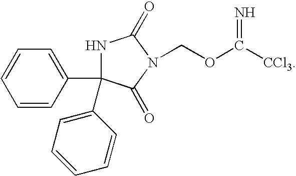

6. A process for preparing fosphenytoin or a salt thereof, comprising reacting a compound having a formula: 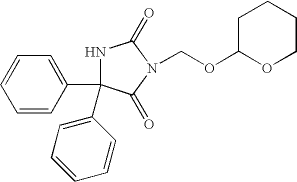
with a benzyl halide, to from a compound having a formula:

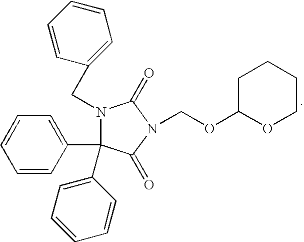

7. The process of claim 6, wherein a compound having a formula: 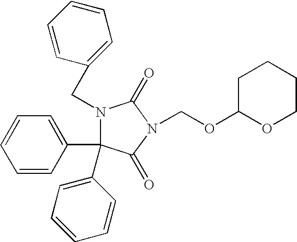
is prepared by reacting a compound having a formula: 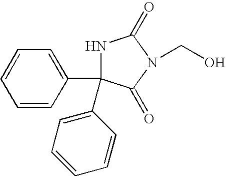
with 3,4-dihydro-2H-pyran.

8. A process for preparing fosphenytoin or a salt thereof, comprising reacting a compound having a formula: 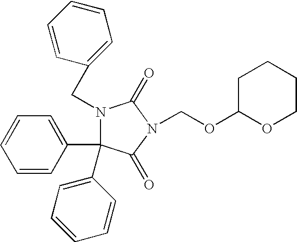
with an acid to form a compound having a formula:

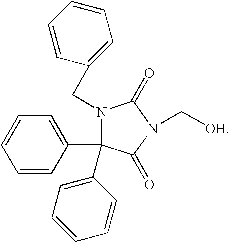

9. A process for preparing fosphenytoin or a salt thereof, comprising reacting a compound having a formula: 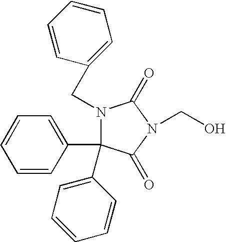
with a halogenating agent to form a compound having a formula: 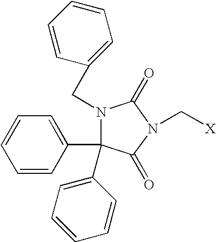
where X is Cl, Br, I.

10. A process for preparing fosphenytoin or a salt thereof, comprising reacting a compound having a formula: 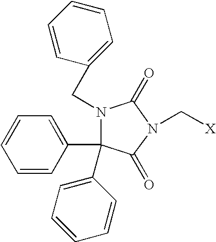
with a dibenzyl phosphate to form a compound having a formula:

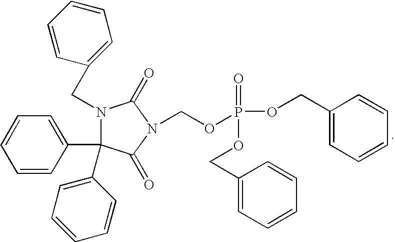

11. A process for preparing fosphenytoin or a salt thereof, comprising reacting a compound having a formula: 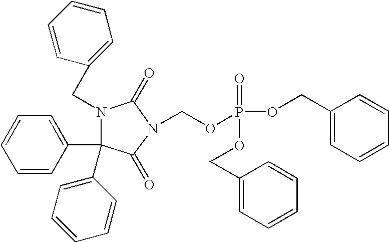
with a reducing agent to form fosphenytoin.

12. A compound having a formula:

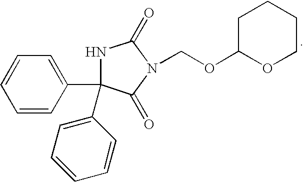

13. A compound having a formula:

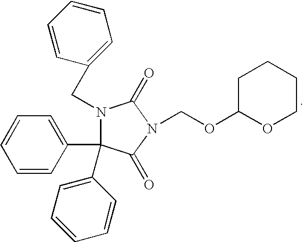

14. A compound having a formula:

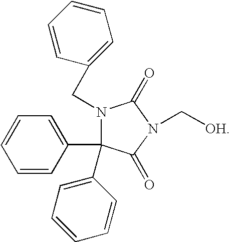

15. A compound having a formula: 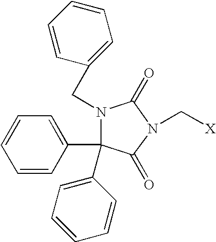
where X is Cl, Br, I.

16. A compound having a formula:

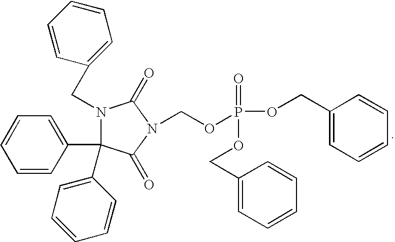

17. Fosphenytoin or a salt thereof, containing less than about 0.5 weight percent of individual impurities comprising one or more of:
a) a compound having a formula: 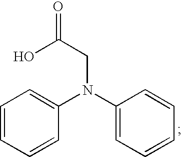
b) a compound having a formula: 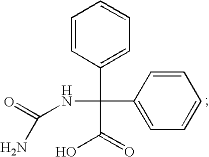
c) a compound having a formula: 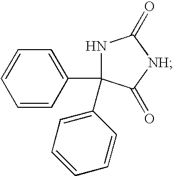
d) a compound having a formula: 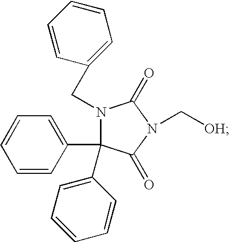
e) a compound having a formula: 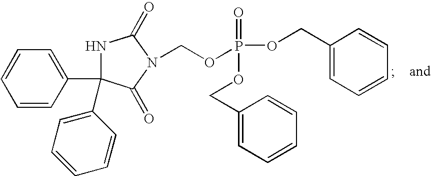
f) a compound having a formula:

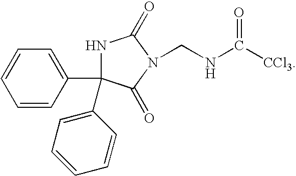

18. Fosphenytoin or a salt thereof, containing less than less than about 2 weight percent of a phosphate salt.

19. A process for converting fosphenytoin to a disodium salt, comprising reacting fosphenytoin with about 2 to about 2.5 moles of sodium hydroxide, per mole of fosphenytoin.
